(19) United States
(12) Patent Application Publication
(10) Pub. No.: US 2024/0181236 A1
(43) Pub. Date: Jun. 6, 2024

(54) HEMOSTASIS SEAL WITH LOW PASS-THROUGH FRICTION FORCE

(71) Applicant: EDWARDS LIFESCIENCES CORPORATION, Irvine, CA (US)

(72) Inventors: Hsingching C. Hsu, Santa Ana, CA (US); Pablo Hernan Catania, Costa Mesa, CA (US)

(21) Appl. No.: 18/443,984

(22) Filed: Feb. 16, 2024

Related U.S. Application Data
(63) Continuation of application No. PCT/US2022/039335, filed on Aug. 3, 2022.
(60) Provisional application No. 63/234,660, filed on Aug. 18, 2021.

(51) Int. Cl. A61M 39/06

(52) U.S. Cl. CPC A61M 39/0606; A61M 2039/062; A61M 2039/0633

(57) ABSTRACT

Disclosed herein are hemostasis seals with low pass-through friction force. The disclosed seals are configured for use with transcatheter delivery systems or vascular access sheath systems. The seals are a unitary design that provide low pass-through friction when passing a catheter through the seal. The specifics of the design that provides the desirable low pass-through friction include the physical configuration of the seals and the material used for the seals. The disclosed seals include an outer portion, an inner annular portion, and a webbing portion connecting the outer portion to the inner annular portion, forming a suspended O-ring. The suspended O-ring provides an opening through which a delivery device can be passed and applies a relatively low friction force on the delivery device as it is passed through the opening.

### CROSS-REFERENCE TO RELATED APPLICATIONS

This application is a continuation of International Patent Application No. PCT/US2022/039335, filed on Aug. 3, 2022 and entitled HEMOSTASIS SEAL WITH LOW PASS-THROUGH FRICTION FORCE, which claims the benefit of priority to U.S. Prov. App. No. 63/234,660, filed on Aug. 18, 2021 and entitled HEMOSTASIS SEAL WITH LOW PASS-THROUGH FRICTION FORCE, the complete disclosures of which are hereby incorporated by reference in their entireties for all purposes.

### BACKGROUND

### Field

The present disclosure relates generally to the field of sealing devices that provide a leak-resistant seal in catheter delivery systems and/or vascular access devices.

### Description of Related Art

Inserting a delivery device, such as an interventional catheter device, into the vascular system typically involves a needle stick followed by dilating a blood vessel (e.g., artery or vein). An introducer sheath is inserted into the dilated blood vessel and left in place for the duration of the procedure. The introducer sheath acts as the main conduit for entry of subsequent therapeutic or diagnostic devices.

Typically, introducer sheaths contain a hemostatic component that restricts back-flow of blood from the blood vessel. The hemostatic component (e.g., a hemostasis seal) is generally passive and provides sealing around the catheter devices and guide wires that are used during the procedure. In some cases, hemostasis seals include a structure through which a device such as a guide wire or other medical device component is inserted. The hemostasis seals are configured to maintain hemostasis while allowing medical devices to be passed through the seals.

### SUMMARY

For purposes of summarizing the disclosure, certain aspects, advantages and novel features have been described herein. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

In an aspect, the present disclosure provides a hemostatic seal for use in a transcatheter delivery systems or vascular sheath access system. The hemostatic seal is designed to have a low pass-through friction force while still providing sealing around a tool passed through the hemostatic seal.

In some implementations, the hemostatic seal includes an outer portion with a first cross-section thickness. In some implementations, the hemostatic seal includes an inner annular portion with a second cross-section thickness. In some implementations, the hemostatic seal includes a thin web portion with a third cross-section thickness, the thin web portion connecting the inner annular portion to the outer portion. In some implementations, the first cross-section thickness is greater than the second cross-section thickness and the second cross-section thickness is greater than the third cross-section thickness.

In some implementations, the outer portion, the inner annular portion, and the thin web portion are made from a single, unitary piece of material. In some implementations, the material is a low durometer elastomer.

In some implementations, the inner annular portion forms a central hole through which a delivery device is configured to pass. In some implementations, passing a delivery device through the inner annular portion causes the inner annular portion to move relative to the outer portion. In some implementations, increasing the third cross-section thickness reduces movement of the inner annular portion responsive to passing the delivery device through the inner annular portion.

In some implementations, increasing the second cross-section thickness increases resistance of the inner annular portion to radial expansion. In some implementations, the outer portion has a substantially circular cross-section. In some implementations, the inner annular portion has a substantially circular cross-section. In some implementations, the third cross-section thickness is substantially uniform between the outer portion and the inner annular portion.

In some implementations, the present disclosure provides an access sheath. In some implementations, the access sheath includes a tubular shaft having a proximal end and a distal end. In some implementations, the access sheath includes a hub that is adapted for coupling to the proximal end of the tubular shaft. In some implementations, the access sheath includes a first hemostatic seal configured to prevent leakage through the introducer sheath. In some implementations, the first hemostatic seal comprises an outer portion with a first cross-section thickness, an inner annular portion with a second cross-section thickness, and a thin web portion with a third cross-section thickness, the thin web portion connecting the inner annular portion to the outer portion, the first cross-section thickness is greater than the second cross-section thickness and the second cross-section thickness is greater than the third cross-section thickness.

In some implementations, the first hemostatic seal is removably positioned at a proximal portion of the hub.

In some implementations, the access sheath further includes a second hemostatic seal. In some implementations, the second hemostatic seal is adjacent to the first hemostatic seal. In some implementations, the second hemostatic seal comprises an outer portion, an inner annular portion, and a thin web portion that connects the inner annular portion to the outer portion, wherein a cross-section thickness of the outer portion is greater than a cross-section thickness of the inner annular portion and a cross-section thickness of the inner annular portion is greater than a cross-section thickness of the thin web portion.

In some implementations, the first hemostatic seal is made from a single, unitary piece of material. In some implementations, the third cross-section thickness is substantially uniform between the outer portion and the inner annular portion. In some implementations, the inner annular portion forms a central hole through which a delivery device is configured to pass.

In some implementations, passing a delivery device through the hub causes the delivery device to pass through the inner annular portion which causes the inner annular portion to move relative to the outer portion. In some implementations, increasing the third cross-section thickness reduces movement of the inner annular portion responsive to passing the delivery device through the inner annular portion.

### BRIEF DESCRIPTION OF THE DRAWINGS

Various embodiments are depicted in the accompanying drawings for illustrative purposes and should in no way be interpreted as limiting the scope of the disclosed embodiments. In addition, features of different disclosed implementations can be combined to form various embodiments, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of aspects thereof.

FIGS. 1A, 1B, and 1C illustrate an example introducer sheath with which various implementations of the disclosed seals may be used.

FIGS. 2A-D illustrate a top view, a side view, a cross-section view, and an elevated view of an example seal, respectively, the seal configured to maintain hemostasis in various medical devices, such as access sheaths and loader devices.

FIG. 3 illustrates a cross-section view of an example seal to demonstrate dimensions of the seal.

FIGS. 4A and 4B illustrate an example seal with a delivery device being passed through a central hole formed by an inner annular portion of the seal.

FIGS. 5A and 5B illustrate top views of example seals with ribs incorporated into the thin web portion.

### DETAILED DESCRIPTION

The headings provided herein are for convenience only and do not necessarily affect the scope or meaning of the claimed subject matter.

### Overview

Introducer sheaths can be used in various clinical procedures to facilitate the use of delivery devices, such as interventional catheter devices. The introducer sheath is inserted into a dilated blood vessel and left in place for the duration of the clinical procedure. Introducer sheaths typically contain a hemostatic component (e.g., a hemostasis seal) that restricts back-flow of blood from the blood vessel. One of the main functions of hemostasis seals is to provide hemostasis to prevent blood loss during the clinical procedure. In some delivery systems, such as vascular access sheath systems, components of the delivery system may actively pass through one or more seals. In such systems, it would be desirable to have seals with a relatively low friction force that are also able to maintain hemostasis.

For access sheath systems in particular, the force necessary to pass catheters through the sheath seals is typically high. This creates particular challenges that include, for example, potential damage to catheters passing through the seals, undesirable ergonomic forces required to push catheters through the seals, and reduced tactile feedback when pushing catheters through the seals.

Accordingly, disclosed herein are hemostasis seals with low pass-through friction force. The disclosed seals are configured for use with transcatheter delivery systems or vascular access sheath systems. The seals are a unitary design designed to provide low pass-through friction when passing a catheter through the seal. The specifics of the design that provides the desirable low pass-through friction include the physical configuration of the seals and/or the material used for the seals. The disclosed seals include an outer ring portion, an inner ring portion, and a webbing portion connecting the outer ring portion to the inner ring portion. The disclosed seals include a suspended O-ring, or an O-ring suspended by webbing, a web, or a membrane. The suspended O-ring provides an opening through which a delivery device can be passed and applies a relatively low friction force on the delivery device as it is passed through the opening.

The disclosed seals are particularly useful in loader accessories, which can be used with transcatheter shunt delivery systems, for example. The disclosed seals are configured to maintain hemostasis and to have a relatively low friction force (e.g., for devices passing through the seal). The low friction force advantageously allows the user to have tactile feedback of the delivery system during a clinical procedure. The disclosed seals can also be configured to accommodate a variety of different types and sizes of delivery catheters to maintain hemostasis while having a relatively low friction force as the delivery catheter is inserted through the seal. In some implementations, one or more of the disclosed hemostasis seals can be used together (e.g., in series along the longitudinal axis) to improve hemostasis in delivery systems and/or sheath systems.

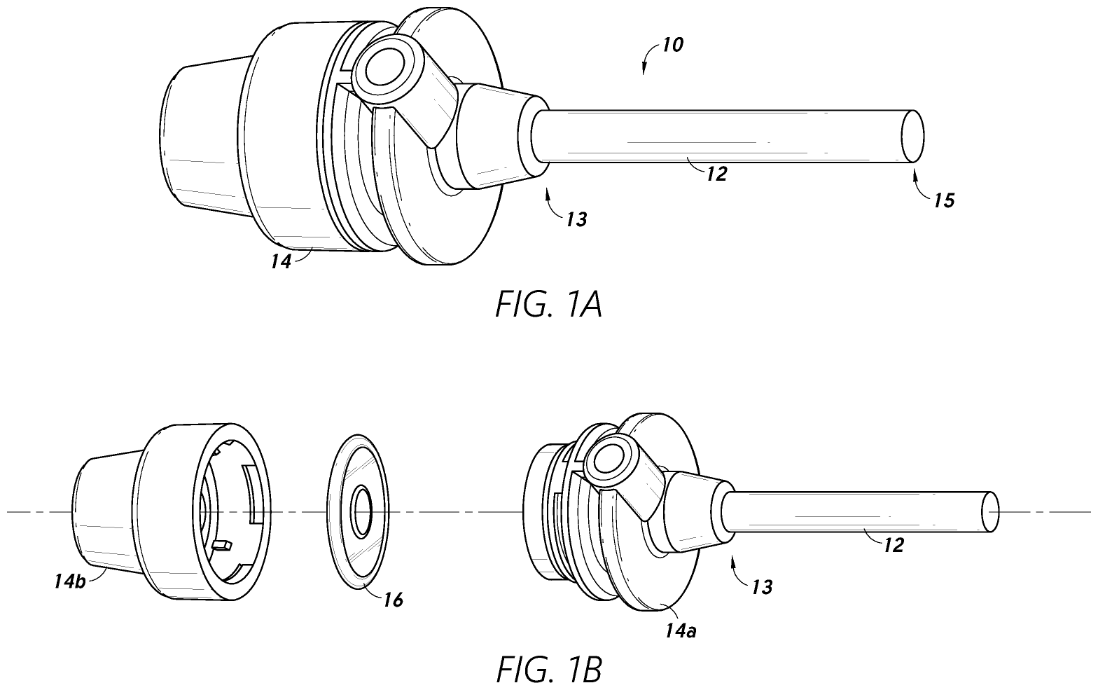

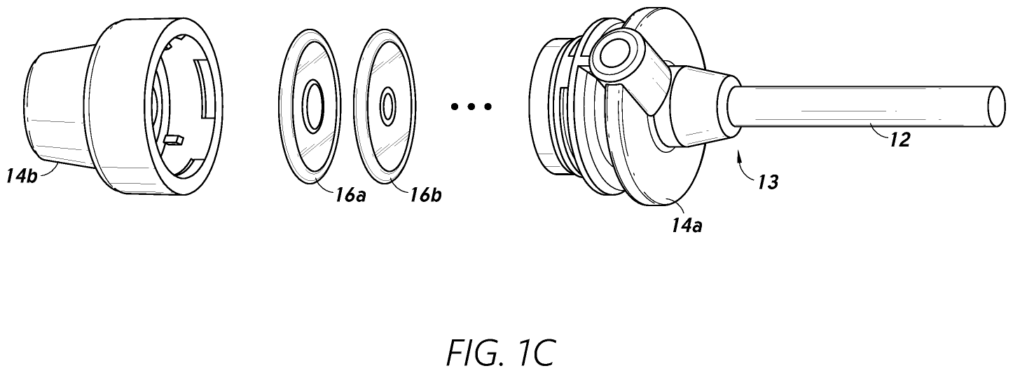

FIGS. 1A, 1B, and 1C illustrate an example introducer sheath 10 with which various implementations of the disclosed seals may be used. The introducer sheath 10 generally comprises a tubular shaft 12 having a proximal end 13 and a distal end 15 and a two-piece hub 14 that is adapted for coupling to the proximal end 13 of shaft 12. Hub 14 comprises hub main body 14a and cap 14b which may be coupled to one another by any appropriate means, such as by threading or, as shown in FIGS. 1A-1C, by being press fit or mechanically snapped into place.

FIG. 1B illustrates an exploded view of the introducer sheath 10 to show a hemostatic seal 16, examples of which are described in more detail herein. The seal 16 is configured to form a hemostatic seal to prevent leakage through the introducer sheath 10 when a delivery device is inserted through the introducer sheath 10 at an access site of a patient. The hemostatic seal 16 may be removably positioned at a proximal portion 13 of the introducer sheath 10 within the hub 14. In some implementations, the seal 16 may be positioned at any desired portion of the sheath 10. It will be appreciated that the depictions herein are for illustrative purposes only and do not necessarily reflect the actual shape, size, or dimensions of the sheath 10 or seal 16. For example, the shaft 12 has a greater length than illustrated in FIGS. 1A-1C.

In some implementations, the seal 16 is attached to hub 14. The seal 16 can be attached to the hub 14 in one of various manners, for example, via an adhesive or other bonding means. The seal 16 includes a centrally located opening through which a delivery device can pass. The seal 16 facilitates hemostasis through the introducer sheath 10 during a surgical or other medical procedure. Other seals (not shown) can also be arranged through the introducer sheath 10, as needed or desired, for maintaining hemostasis. It is to be understood that the seal 16 can be used with other devices besides introducer sheaths such as, for example, loader devices.

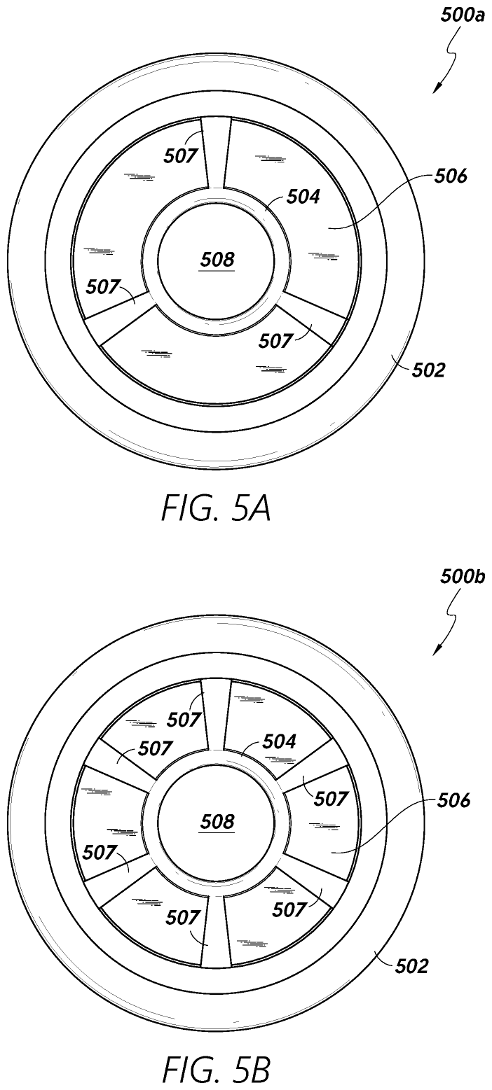

FIG. 1C illustrates an exploded view of the introducer sheath 10 to show that two or more hemostatic seals 16a, 16b can be used in series within the hub 14 to improve hemostatic sealing of the introducer sheath 10. In some implementations, the seals 16a, 16b can be dissimilar (e.g., different seals can have different dimensions of an inner annular portion and/or a thin web portion or may contain no ribs or a different number of ribs, examples of which are described with reference to FIGS. 5A and 5B). In some implementations, two or more seals 16a, 16b have identical or nearly identical properties. The seals 16a, 16b can be combined to provide targeted sealing and friction force properties to improve performance and feel (e.g., tactile feedback during insertion of a delivery device) during procedures performed with the introducer sheath 10. In some implementations, the seals 16a, 16b are adjacent to one another. In general, increasing the number of seals 16 increases hemostasis functionality and increases friction. In some implementations, using two or more seals 16 can be advantageous in situations where a delivery device is being passed through in a way that deviates from a central longitudinal axis (e.g., it is off-center relative to the introducer sheath 10). In such situations, multiple seals 16 can prevent leaks caused by non-concentric or off-center delivery device insertion.

### Example Hemostatic Seals

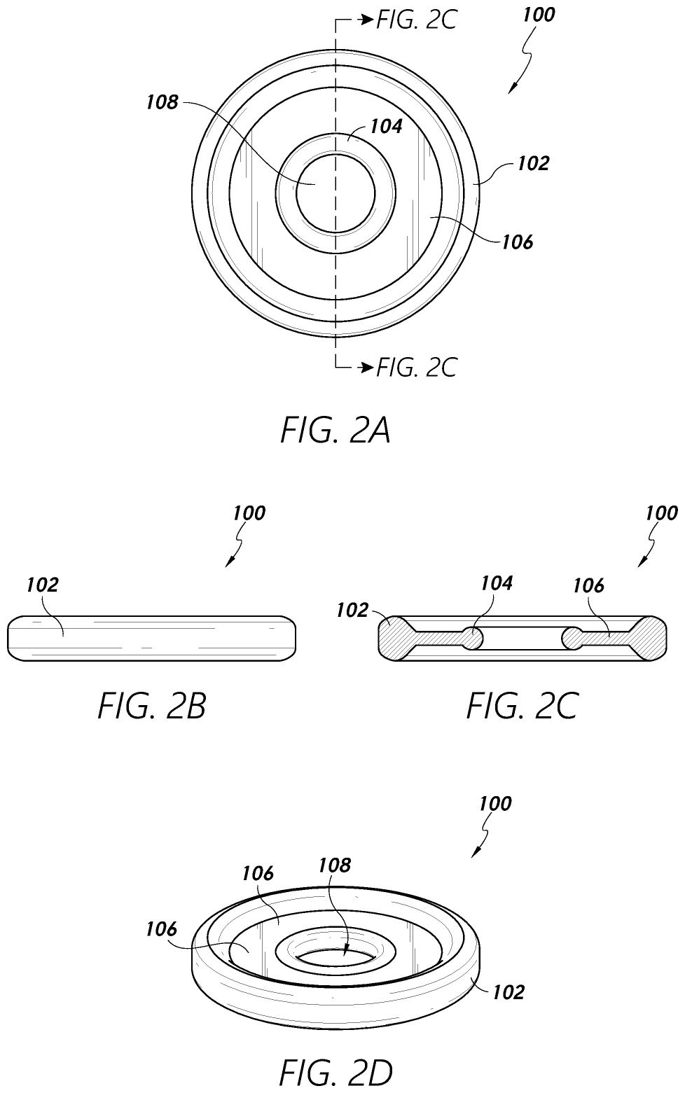

FIGS. 2A-D include a top view, a side view, a cross-section view, and an elevated view of an example seal 100, the seal 100 configured to maintain hemostasis in various medical devices, such as access sheaths and loader devices. The seal 100 is similar to the seals 16, 16a, 16b described herein with reference to FIGS. 1A-1C. The seal 100 is a disk structure with an outer portion 102 (e.g., an O-ring type shape) and an inner annular portion 104 (e.g., a seal gland) connected to the outer portion 102 by a thin web portion 106 (or membrane). The thin web portion 106 is solid in the sense that there are no discontinuities, perforations, or the like in the thin web portion 106. The thickness of the cross-section of the outer portion 102 is greater than the thickness of the cross-section of the inner annular portion 104. The thickness of the cross-section of the thin web portion 106 connecting the outer portion 102 and the inner annular portion 104 is less than the thickness of the cross-section of the inner annular portion 104. The seal 100 is made from a single piece of material.

The inner annular portion 104 forms a central hole 108 through which a delivery device, medical instrument, guidewire, etc. can be passed. The central hole 108 or orifice is configured to accommodate and provide a fluid-tight seal around medical instruments inserted through the seal 100. The diameter of the central hole 108 can be configured to be about 0.010 inches smaller than a targeted delivery device to be used with the seal, about 0.007 inches smaller, about 0.005 inches smaller, or about 0.003 inches smaller. The diameter of the central hole 108 is configured to be smaller than an outer diameter of the delivery device to provide a fluid-tight seal. The central hole 108 may be expansible from a first diameter when no medical instrument traverses the sealing aperture (e.g., before or after instrument insertion) to a second diameter (larger than the first diameter) when the medical instrument traverses through the central hole 108 formed by the inner annular portion 104 (e.g., during instrument insertion, placement or withdrawal). In some implementations, the inner annular portion 104 provides uninterrupted contact around an outer surface of the instrument inserted through the central hole 108. This may be due at least in part to the expansible property of the inner annular portion 104.

The seal 100 can be generally formed from a low durometer elastomer. Examples of low durometer elastomers include, for example and without limitation, silicone or any elastomeric polymer, rubber, a combination thereof, or like medical device grade materials that can accommodate a variety of medical instrument diameters without plastic deformation or tearing. A useful material of the seal 100 comprises a silicone material (e.g., elastosil 3003/60 A/B) with a rubber durometer of about Shore 60 A.

The combination of the material and the design (the outer portion 102, the inner annular portion 104, and the thin web portion 106 connecting the outer portion 102 and the inner annular portion 104 with the various dimensions of these portions) allows the disclosed seals to provide sufficient hemostasis to reduce or minimize blood loss and to reduce the friction force for catheters or other instruments that pass through the disclosed seals. Advantages of having low friction include reducing the risk of damage to the passing catheter, allowing for tactile feedback during the clinical procedure, and providing an acceptable ergonomic catheter insertion/removal force through the seal 100. Low friction through the seal 100 can be advantageous compared to higher friction seals which tend to swamp tactile feedback to the clinician during insertion and maneuvering of a delivery device. The low friction of the disclosed seals allows tactile feedback caused by the delivery device pressing against tissue in the patient. In addition, the low pass-through friction seals disclosed herein also provide hemostasis. The disclosed seals are a passive, unitary or integral (e.g., one-piece) component design. This is different from other seals that include multiple components and/or require activation to provide hemostasis and low friction (e.g., balloon inflated seals).

The inner annular portion 104 is designed to be similar to or approximate a floating O-ring supported by the thin web portion 106. The outer portion 102 can be similarly designed to be similar to or approximate an O-ring, similar to a gland, and can be configured to provide sealing between the seal 100 and the housing of the device in which it is used. This sealing provided by the outer portion 102 can be compared to the inner annular portion 104 that provides sealing between the seal 100 and a delivery device inserted through the seal 100. The outer portion 102 is configured to provide compression within the housing and can absorb compression forces applied by the housing to support the thin web portion 106 and the inner annular portion 104. The thin web portion 106 acts to support the inner annular portion 104 so that the inner annular portion 104 floats (e.g., has some freedom to move relative to the outer portion 102). The floating inner seal provided by the inner annular portion 104 allows the inner annular portion 104 to move relative to the outer portion 102 when a delivery device is passed through the seal 100. The thickness of the inner annular portion 104 and the thickness of the thin web portion 106 affects the friction force on the delivery device being passed through the seal 100. Thus, the thickness of each component (e.g., the thin web portion 106 and the inner annular portion 104) can be tailored to achieve a desired or targeted friction force and sealing functionality. These parameters may also be adjusted depending at least in part on a delivery device to be passed through the seal 100. The targeted friction force can be a force such that forces experienced by the delivery device due to interaction with the tissue of the patient and applied by the clinician are larger than the friction force resisting movement of the delivery device through the seal 100. In some implementations, the relatively low pass-through friction force is due at least in part to the thicknesses of the inner annular portion 104, the thin web portion 106, and/or the material of the seal 100.

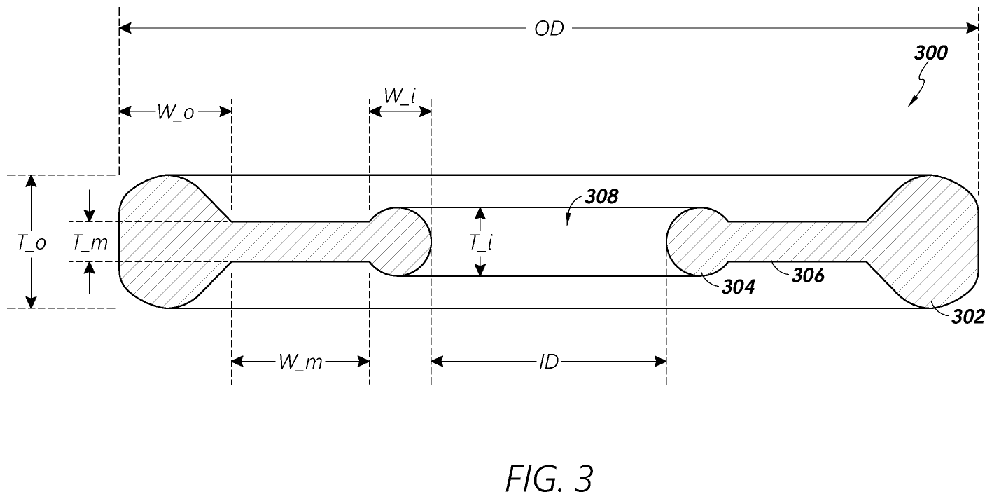

FIG. 3 illustrates a cross-section view of an example seal 300 to demonstrate dimensions of the seal 300. The seal 300 is similar to the seal 100 described herein with reference to FIGS. 2A-D and the seals 16, 16a, 16b described herein with reference to FIGS. 1A-1C. The seal 300 includes an outer portion 302 and an inner annular portion 304 connected by a thin web portion 306, the inner annular portion 304 forming a central hole 308. The outer portion 302 has a cross-section thickness, T_o, and a cross-section width, W_o. In some implementations, the outer portion 302 has an approximately or substantially circular cross-section such that the thickness, T_o, is a measure of a diameter of the circular cross-section. Similarly, the inner annular portion 304 has a cross-section thickness, T_i, and a cross-section width, W_i. In some implementations, the inner annular portion 304 has an approximately or substantially circular cross-section such that the thickness, T_i, is a measure of a diameter of the circular cross-section. The thin web portion 306 has a cross-section thickness, T_m. The thin web portion 306 extends from the outer portion 302 to the inner annular portion 304, representing a radial distance or width, W_m. The outer portion thickness, T_o, is greater than the inner portion thickness, T_i, which is greater than the web portion thickness, T_m (T_o>T_i>T_m). The inner diameter of the inner annular portion, ID, forms the outer diameter of the central hole 308. The inner diameter, ID, of the inner annular portion 304 can increase due to the elasticity of the material used to form the seal 300. This enables the formation of a fluid-tight seal around a device inserted through the central hole 308 formed by the inner annular portion 304. In some implementations, the cross-section thickness of the thin web portion 306 is substantially uniform between the outer portion 302 and the inner annular portion 304.

In some implementations, the central hole 308 or inner diameter, ID, of the inner annular portion 304 is designed to accommodate a delivery device of a particular size. For example, the inner diameter, ID, can be a couple thousandths of an inch smaller than an outer diameter of the delivery device, as described herein. In some implementations, the outer diameter, OD, of the seal 300 is designed to be compatible and/or fit in a particular access device, such as an introducer sheath. For example, the outer diameter, OD, can be sized to fit within a housing and/or have the outer portion 302 be compressed longitudinally by components within a housing or that are part of the housing to form a hemostatic seal.

In certain implementations, the seal 300 is designed for use in venous access procedures due at least in part to lower pressures experienced in venous access procedures. In some implementations, the thicknesses and/or cross-sections of the thin web portion 306 and/or the inner annular portion 304 can be increased relative to seals designed for venous access procedures to accommodate higher pressures experienced in arterial access procedures.

The thicknesses of the various components affect the performance of the seal 300. For example, the thickness of the inner annular portion 304 (e.g., T_i, W_i) affects how well the inner diameter is able to expand radially outward. For example, increasing the cross-section thickness of the inner annular portion 304 can increase the resistance of the inner annular portion 304 to radial expansion. In general, the thicker the cross-section of the inner annular portion 304 the more resistance there is to radially outward expansion. Conversely, thinner cross-sections of the inner annular portion 304 correspond to less resistance to outward radial expansion of the inner diameter, ID. The smaller the inner diameter, ID, relative to the outer diameter of the delivery device, the more resistance or friction force will be felt passing the delivery device through the seal 300. In some implementations, increasing the thickness of the thin web portion 306 reduces movement of the inner annular portion 304 responsive to passing a delivery device through the central hole 308 formed by the inner annular portion 304.

In some implementations, the thickness of the outer portion 302, T_o, can be about 0.100 inches or between about 0.090 inches and about 0.110 inches, between about 0.070 inches and about 0.140 inches, or between about 0.050 inches and about 0.200 inches. In some implementations, the width of the outer portion 302, W_o, can be about 0.075 inches or between about 0.070 inches and about 0.100 inches, between about 0.060 inches and about 0.110 inches, or between about 0.050 inches and about 0.150 inches.

In some implementations, the thickness of the inner annular portion 304, T_i, can be about 0.050 inches or between about 0.040 inches and about 0.060 inches, between about 0.030 inches and about 0.070 inches, or between about 0.025 inches and about 0.100 inches. In some implementations, the width of the inner annular portion 304, W_i, can be about 0.045 inches or between about 0.040 inches and about 0.060 inches, between about 0.030 inches and about 0.070 inches, or between about 0.025 inches and about 0.100 inches. In some implementations, the inner annular portion 304 has a circular cross section with a radius of about 0.025 inches or between about 0.020 inches and about 0.030 inches, between about 0.015 inches and about 0.040 inches, or between about 0.010 inches and about 0.050 inches.

In some implementations, the inner diameter central hole 308, ID, is about 0.190 inches or between about 0.175 inches and about 0.200 inches, between about 0.150 inches and about 0.250 inches, or between about 0.100 inches and about 0.400 inches. In certain implementations, the inner diameter of the central hole 308, ID, is configured to be a couple thousandths of an inch (e.g., about 0.002 inches) smaller than a particular delivery device or delivery devices having a particular size. In some implementations, the outer diameter of the seal 300, OD, is about 0.690 inches or between about 0.600 inches and about 0.800 inches, between about 0.500 inches and about 0.900 inches, or between about 0.400 inches and about 1.000 inches. In certain implementations, the outer diameter of the seal 300, OD, is configured to be compatible with a particular access device or delivery devices having a particular size.

In some implementations, the thickness of the web portion 306, T_m, can be about 0.030 inches or between about 0.020 inches and about 0.040 inches, between about 0.015 inches and about 0.050 inches, or between about 0.010 inches and about 0.090 inches. In some implementations, the width of the web portion 306, W_m, can be about 0.240 inches or between about 0.200 inches and about 0.250 inches, between about 0.150 inches and about 0.300 inches, or between about 0.125 inches and about 0.400 inches.

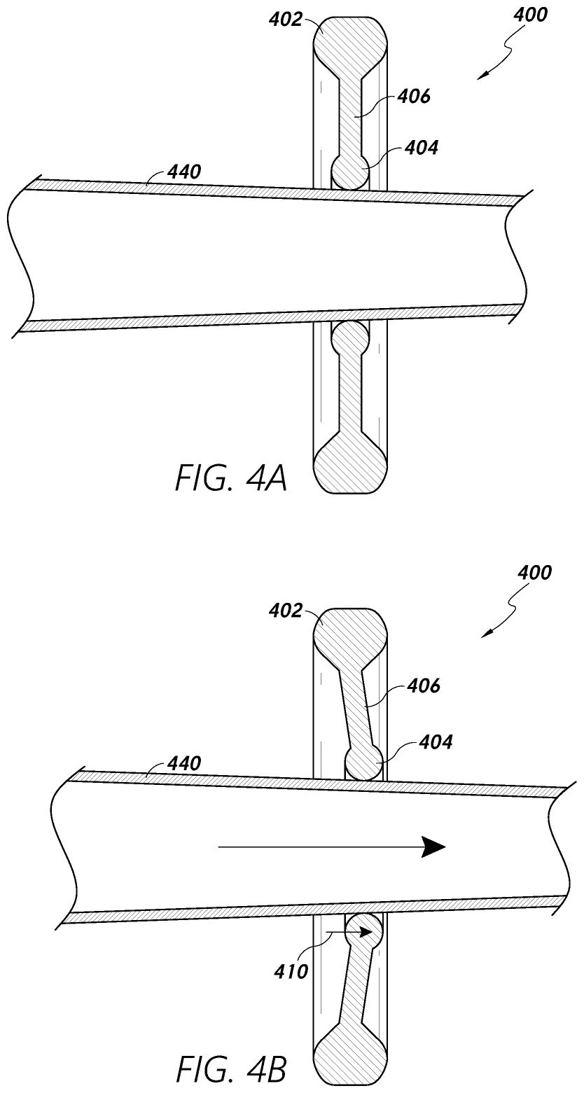

The thickness of the web portion 306, T_m, affects the amount of flexion experience by the thin web portion 306 as a catheter passes through the central hole 308, an example of which is illustrated in FIGS. 4A and 4B. FIGS. 4A and 4B illustrate an example seal 400 (e.g., within an introducer sheath, not shown) with a delivery device 440 being passed through a central hole formed by an inner annular portion 404 of the seal 400. The seal 400 is similar to the seals described herein such as seals 16, 16a, 16b, 100, 300. The seal 400 includes an outer portion 402 and a web portion 406 that connects the inner annular portion 404 to the outer portion 402. The seal 400 and the delivery device 440 are illustrated in a cross-section view to illustrate flexion of the seal 400 among other things.

First, as the delivery device 440 is introduced through the central hole formed by the inner annular portion 404, the inner annular portion 404 expands radially outward to accommodate the delivery device 440. The contact between the inner annular portion 404 and the outer surface of the delivery device 440 provides hemostasis and applies a friction force on the delivery device 440. As the delivery device 440 passes through the inner annular portion 404, the web portion 406 flexes in the direction the delivery device 440 is being pushed (illustrated by the large arrow in FIG. 4B), where the flexion of the web portion 406 is illustrated by the little arrow 410. This deflection is experienced as resistance to movement of the delivery device 440, or a friction force. The amount of flex corresponds to a thickness of the web portion 406. The thicker the web portion 406, the less flexion that occurs resulting in a greater resistance to the delivery device 440 as it is advanced or retracted, experienced as a friction force by the clinician. Conversely, a thinner web portion 406 results in less resistance to movement of the inner annular portion 404 (e.g., movement relative to the outer portion 402 which is relatively fixed to the housing of the introducer sheath or other access device).

A hemostatic seal can be achieved as the delivery device 440 passes through the seal 400. The design of the seal 400 affects the resistance to movement of the delivery device 440 and can be configured to provide a targeted friction force (e.g., the targeted friction force is less than typical forces arising due to interactions between the delivery device 440 and tissue of the patient) and to provide sufficient hemostatic sealing.

In some implementations, the friction force is about 1.54 N, or between about 1.2 N and about 1.90 N, or between about 1 N and about 2 N, or between about 0.9 N and about 3.0 N. This friction force can correspond to the insertion force of the deliver device 440 through the seal 400, the insertion force through the access device, or the force to insert the delivery device 440 into the access device and through the loader.

FIGS. 5A and 5B illustrate top views of example seals 500a, 500b with ribs 507 incorporated into a thin web portion 506. The seals 500a, 500b are similar to the seals 16, 16a, 16b, 100, 300, 400 described herein and thus include an outer portion 502, a web portion 506, and an inner annular portion 504 forming a central hole 508. The seal 500a includes three ribs 507 aligned radially from the inner annular portion 504 to the outer portion 502, or partially from the inner annular portion 504 to the outer portion 502. The ribs 507 form part of the web portion 506. The ribs 507 comprise areas of the web portion 506 that are thicker than the remaining area of the web portion 506. The seal 500b includes six ribs 507 configured similarly to the ribs 507 of the seal 500a.

The number of ribs 507 and the dimensions of the ribs 507 affect the pliability or stiffness of the web portion 506. This can improve hemostasis capabilities while increasing the friction forces of delivery devices passing through the seals 500a, 500b. The number of ribs can vary and may be less than three, more than three and less than six, or more than six.

### Additional Description of Examples

Example 1: A hemostatic seal for use in a transcatheter delivery systems or vascular sheath access system. the hemostatic seal includes an outer portion with a first cross-section thickness; an inner annular portion with a second cross-section thickness; and a thin web portion with a third cross-section thickness, the thin web portion connecting the inner annular portion to the outer portion, wherein the first cross-section thickness is greater than the second cross-section thickness and the second cross-section thickness is greater than the third cross-section thickness.

Example 2: The hemostatic seal of any example herein, in particular example 1, wherein the outer portion, the inner annular portion, and the thin web portion are made from a single, unitary piece of material.

Example 3: The hemostatic seal of any example herein, in particular example 2, wherein the material is a low durometer elastomer.

Example 4: The hemostatic seal of any example herein, in particular examples 1-3, wherein the inner annular portion forms a central hole through which a delivery device is configured to pass.

Example 5: The hemostatic seal of any example herein, in particular examples 1-4, wherein passing a delivery device through the inner annular portion causes the inner annular portion to move relative to the outer portion.

Example 6: The hemostatic seal of any example herein, in particular example 5, wherein increasing the third cross-section thickness reduces movement of the inner annular portion responsive to passing the delivery device through the inner annular portion.

Example 7: The hemostatic seal of any example herein, in particular examples 1-6, wherein increasing the second cross-section thickness increases resistance of the inner annular portion to radial expansion.

Example 8: The hemostatic seal of any example herein, in particular examples 1-7, wherein the outer portion has a substantially circular cross-section.

Example 9: The hemostatic seal of any example herein, in particular examples 1-8, wherein the inner annular portion has a substantially circular cross-section.

Example 10: The hemostatic seal of any example herein, in particular examples 1-9, wherein the third cross-section thickness is substantially uniform between the outer portion and the inner annular portion.

Example 11: An access sheath that includes a tubular shaft having a proximal end and a distal end; a hub that is adapted for coupling to the proximal end of the tubular shaft; and a first hemostatic seal configured to prevent leakage through the access sheath, the first hemostatic seal comprising an outer portion with a first cross-section thickness, an inner annular portion with a second cross-section thickness, and a thin web portion with a third cross-section thickness, the thin web portion connecting the inner annular portion to the outer portion, the first cross-section thickness is greater than the second cross-section thickness and the second cross-section thickness is greater than the third cross-section thickness.

Example 12: The access sheath of any example herein, in particular example 11, wherein the first hemostatic seal is removably positioned at a proximal portion of the hub.

Example 13: The access sheath of any example herein, in particular examples 11-12 further comprising a second hemostatic seal.

Example 14: The access sheath of any example herein, in particular example 13, wherein the second hemostatic seal is adjacent to the first hemostatic seal.

Example 15: The access sheath of any example herein, in particular example 13, wherein the second hemostatic seal comprises an outer portion, an inner annular portion, and a thin web portion that connects the inner annular portion to the outer portion, wherein a cross-section thickness of the outer portion is greater than a cross-section thickness of the inner annular portion and a cross-section thickness of the inner annular portion is greater than a cross-section thickness of the thin web portion.

Example 16: The access sheath of any example herein, in particular examples 11-15, wherein the first hemostatic seal is made from a single, unitary piece of material.

Example 17: The access sheath of any example herein, in particular examples 11-16, wherein the third cross-section thickness is substantially uniform between the outer portion and the inner annular portion.

Example 18: The access sheath of any example herein, in particular examples 11-17, wherein the inner annular portion forms a central hole through which a delivery device is configured to pass.

Example 19: The access sheath of any example herein, in particular examples 11-18, wherein passing a delivery device through the hub causes the delivery device to pass through the inner annular portion which causes the inner annular portion to move relative to the outer portion.

Example 20: The access sheath of any example herein, in particular example 19, wherein increasing the third cross-section thickness reduces movement of the inner annular portion responsive to passing the delivery device through the inner annular portion.

The term “catheter” is used herein according to its broad and ordinary meaning and may include any tube, sheath, steerable sheath, steerable catheters, and/or any other type of elongate tubular delivery device comprising an inner lumen configured to slidably receive instrumentation, such as for positioning within an atrium or coronary sinus, including for example delivery catheters and/or cannulas.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. The structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

Delivery systems as described herein may be used to position catheter tips and/or catheters to various areas of a human heart. For example, a catheter tip and/or catheter may be configured to pass from the right atrium into the coronary sinus. However, it will be understood that the description can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, such as for positioning within an atrium or coronary sinus, including for example delivery catheters and/or cannulas. It will be understood that other types of medical implant devices and/or procedures can be delivered to the coronary sinus using a delivery system as described herein, including for example ablation procedures, drug delivery and/or placement of coronary sinus leads.

What is claimed is:

1. A hemostatic seal for use in a transcatheter delivery systems or vascular sheath access system, the hemostatic seal comprising:
an outer portion with a first cross-section thickness;
an inner annular portion with a second cross-section thickness; and
a thin web portion with a third cross-section thickness, the thin web portion connecting the inner annular portion to the outer portion,
wherein the first cross-section thickness is greater than the second cross-section thickness and the second cross-section thickness is greater than the third cross-section thickness.

2. The hemostatic seal of claim 1, wherein the outer portion, the inner annular portion, and the thin web portion are made from a single, unitary piece of material.

3. The hemostatic seal of claim 2, wherein the material is a low durometer elastomer.

4. The hemostatic seal of claim 1, wherein the inner annular portion forms a central hole through which a delivery device is configured to pass.

5. The hemostatic seal of claim 1, wherein passing a delivery device through the inner annular portion causes the inner annular portion to move relative to the outer portion.

6. The hemostatic seal of claim 5, wherein increasing the third cross-section thickness reduces movement of the inner annular portion responsive to passing the delivery device through the inner annular portion.

7. The hemostatic seal of claim 1, wherein increasing the second cross-section thickness increases resistance of the inner annular portion to radial expansion.

8. The hemostatic seal of claim 1, wherein the outer portion has a substantially circular cross-section.

9. The hemostatic seal of claim 1, wherein the inner annular portion has a substantially circular cross-section.

10. The hemostatic seal of claim 1, wherein the third cross-section thickness is substantially uniform between the outer portion and the inner annular portion.

11. An access sheath comprising:
a tubular shaft having a proximal end and a distal end;
a hub that is adapted for coupling to the proximal end of the tubular shaft; and
a first hemostatic seal configured to prevent leakage through the access sheath, the first hemostatic seal comprising an outer portion with a first cross-section thickness, an inner annular portion with a second cross-section thickness, and a thin web portion with a third cross-section thickness, the thin web portion connecting the inner annular portion to the outer portion, the first cross-section thickness is greater than the second cross-section thickness and the second cross-section thickness is greater than the third cross-section thickness.

12. The access sheath of claim 11, wherein the first hemostatic seal is removably positioned at a proximal portion of the hub.

13. The access sheath of claim 11 further comprising a second hemostatic seal.

14. The access sheath of claim 13, wherein the second hemostatic seal is adjacent to the first hemostatic seal.

15. The access sheath of claim 13, wherein the second hemostatic seal comprises an outer portion, an inner annular portion, and a thin web portion that connects the inner annular portion to the outer portion, wherein a cross-section thickness of the outer portion is greater than a cross-section thickness of the inner annular portion and a cross-section thickness of the inner annular portion is greater than a cross-section thickness of the thin web portion.

16. The access sheath of claim 11, wherein the first hemostatic seal is made from a single, unitary piece of material.

17. The access sheath of claim 11, wherein the third cross-section thickness is substantially uniform between the outer portion and the inner annular portion.

18. The access sheath of claim 11, wherein the inner annular portion forms a central hole through which a delivery device is configured to pass.

19. The access sheath of claim 11, wherein passing a delivery device through the hub causes the delivery device to pass through the inner annular portion which causes the inner annular portion to move relative to the outer portion.

20. The access sheath of claim 19, wherein increasing the third cross-section thickness reduces movement of the inner annular portion responsive to passing the delivery device through the inner annular portion.
